(19) United States
(12) Patent Application Publication
(10) Pub. No.: US 2006/0073123 A1
(43) Pub. Date: Apr. 6, 2006

(54) Adenovirus vectors for immunotherapy

(76) Inventors: Jie Mi, Okemos, MI (US); Andre Lieber, Seattle, WA (US)

Correspondence Address: Jason R Bond;Medlen & Carroll, Suite 350, 101 Howard Street, San Francisco, CA, 94105, US

(21) Appl. No.: 10/511,918

(22) PCT Filed: Apr. 30, 2003
(86) PCT No.: PCT/US03/13560

§ 371 Date: Oct. 11, 2005

(30) Foreign Application Priority Data
Apr. 30, 2002 (US) 60376498

(51) Int. Cl. A61K 48/00; C12N 15/861

(52) U.S. Cl. 424/93.21; 435/456

(57) ABSTRACT

The present invention provides compositions, methods and kits comprising viral vectors that may be used for performing immunotherapy. In particular, the present invention provides viral vectors having subgroup B adenoviral capsid fibers that are configured to express a transgene sequence in antigen presenting cells (e.g. dendritic cells) with a high transduction efficiency. Preferably, the transgene sequence is a retrogen cassette and the adenoviral capsid fibers are Ad11 fibers.

The present Application claims priority to U.S. Provisional Application Ser. No. 60/376,498, filed Apr. 30, 2002, herein incorporated by reference.

Some of the work included in the examples of the present application was funded in part with government support under grant number P01 HL53750 from the National Institute of Health. The government may have certain rights in this invention.

### FIELD OF THE INVENTION

The present invention relates to compositions, methods and kits comprising viral vectors that may be used for performing immunotherapy. In particular, the present invention relates to viral vectors having subgroup B adenoviral capsid fibers that are configured to express a transgene sequence in antigen presenting cells (e.g. dendritic cells) with a high transduction efficiency. Preferably, the transgene sequence is a retrogen cassette and the adenoviral capsid fibers are Ad11 fibers.

### BACKGROUND OF THE INVENTION

Worldwide, the Centers for Disease Control in the US reported that nearly 350 million people are infected with the human hepatitus B virus (HBV). Seventy-five percent of infected individuals live in Asia and the Western Pacific. If not treated effectively, an average of 5 to 10% of HBV infected patients will progress to chronic liver disease. In Asia, the chronic HBV infection rate is 20-30%. In parts of China, 80% of lethal hepatocellular carcinoma (HCC) is due to chronic HBV infections. In the US, about 1 million individuals have chronic HBV infection. As a result of this, about 15,000 die of liver cancer and about 20,000 die from cirrhosis. The causative association between chronic HBV infection and HCC is clearly established (Beasley and Hwang, Semin. Liver Dis., 4:113:21, 1984, herein incorporated by reference). HBV is a noncytophatic virus and liver injury is mainly mediated by the host immune response against virus-infected liver cells and by the production of inflammatory cytokines. A vigorous, polyclonal and multispecific cytotoxic and helper T cell response to HBV is readily detectable in the peripheral blood of patients with acute self limited hepatitis B, but is weak, antigenically restricted or undetectable in patients with chronic infection or HCC (See, Kagawa et al., Cancer Res., 61:3330-8, 2001, herein incorporated by reference.

Because in chronic HBV infection and HBV associated HCC, CTL and Th activity as well as serological immunity against HCV is weak or absent, therapeutic enhancement of the T cell response to HBV core antigens has the potential to terminate chronic HBV infection and attack HCC (See, Ferrari et al., J. Immunol., 145:3442-9, 1990; and Jung et al., J. Virol., 69:3358-68, 1995, both of which are herein incorporated by reference). Several lines of evidence from studies with transgenic mouse-, woodchuck-, or chimpanzee models of HBV infection suggest that HBV immunotherapy is effective in treatment of chronic hepatitis B (See Mancini et al., J. Immunol., 161:5564-70, 1998; and Pancholi et al., Hepatology, 33:448-54, 2001, both of which are herein incorporated by reference). Along this line, two multicenter randomized controlled studies have recently demonstrated the effectiveness of HBV core antigen immunotherapy in decreasing HBV replication and viremia (See Pol et al., J. Hepatol 34:917-21, 2001 and Lau, J. Gastoenterol. Hepatol. 15 Suppl: E46-52, 2000, both of which are herein incorporated by reference. In light of the above, what is need are improved composition and methods for performing immunotherapy that results in the clearance of chronic HBV infection and the elimination of HCC.

### SUMMARY OF THE INVENTION

The present invention provides compositions, methods and kits comprising viral vectors that may be used for performing immunotherapy. In particular, the present invention provides viral vectors having subgroup B adenoviral capsid fibers that are configured to express a transgene sequence in antigen presenting cells (e.g. dendritic cells) with a high transduction efficiency. Preferably, the transgene sequence is a retrogen cassette and the adenoviral capsid fibers are Ad11 fibers.

In some embodiments, the present invention provides compositions comprising an adenoviral vector, wherein the adenoviral vector comprises: a) an adenoviral capsid, wherein the adenoviral capsid comprises subgroup B adenoviral capsid fibers selected from the group consisting of Ad11, Ad14, Ad16, Ad21, Ad34, Ad35, and Ad50; and b) a nucleic acid molecule, wherein the nucleic acid molecule comprises a retrogen cassette sequence encoding a retrogen protein, wherein retrogen protein comprises; i) an antigen protein, ii) a leader sequence (secretory sequence) linked to the N-terminal of the antigen protein, and iii) a cell-binding domain linked to the C-terminal of the antigen protein.

In certain embodiments, the nucleic acid molecule further comprises left and right Ad ITR sequences. In particular embodiments, the nucleic acid molecule further comprises an adenoviral capsid fiber encoding sequence (e.g. encoding adenoviral fibers selected from Ad11, Ad14, Ad16, Ad21, Ad34, Ad35, and Ad50). In additional embodiments, the nucleic acid molecule further comprises an Ad packaging sequence. In some embodiments, the nucleic acid molecule lacks at least one adenoviral gene selected from the group consisting of L5, L2, TP, E1A, E2A, E4, E2A, Pol, IV, E1B, VA, L4, and E3 (e.g. E1 and/or E3). In additional embodiments, the nucleic acid molecule lacks at least five adenoviral genes selected from the group consisting of L5, L2, TP, E1A, E2A, E4, E2A, Pol, IV, E1B, VA, L4, and E3 (e.g. E1, E3, E2A, E2B, and E4). In other embodiments, the nucleic acid molecule lacks all or nearly all of the following adenoviral genes L5, L2, TP, E1A, E2A, E4, E2A, Pol, IV, E1B, VA, L4, and E3 (i.e. “gutless”).

In preferred embodiments, the adenoviral fibers are Ad11 fibers (as shown in the Examples, Ad11 fibers provide greatly improved transduction efficiency, even compared to Ad35 fibers). In additional embodiments, the adenoviral capsid further comprises an Ad5 non-fiber capsid component (i.e. the capsid minus the fibers is Ad5). In some embodiments, the adenoviral capsid further comprises an Ad11 or Ad35 non-fiber capsid component. In particular embodiments, the nucleic acid molecule is inside the adenoviral capsid (See FIG. 1).

In additional embodiments, the antigen protein is a tumor associated antigen. In certain embodiments, the tumor associated antigen is selected from MAGE, GAGE, DAGE, prostate-specific Ag, prostate-specific membrane Ag, tyrosinase, Gp100, α-fetoprotein, Ig idiotype, TCR, Bcr-abl fusion product, mutant p53, NY-ESO-1, Her-2/neu, and Muc-1. In preferred embodiments, the antigen protein is HBeAg or HBcAg (e.g. the adenovirus is used to tranduce dendritic cells, and the dendritic cells are administered to a patient with HBV infection or HCC).

In some embodiments, the cell binding domain comprises an Fc region (e.g. IgG1 Fc region). In other embodiments, the composition further comprises antigen presenting cells (APCs), such as dendritic cells (e.g. immature or mature dendritic cells). In certain embodiments, the composition further comprises an antigen presenting cells (e.g. dendritic cell) and the adenoviral vector is inside the APC (e.g. dendritic cell).

In some embodiments, the present invention provides compositions comprising dendritic cells transduced by the viral vectors of the present invention. Such composition can be administered to patients in need of immunotherapy. In some embodiments, the dendritic cells are originally derived from the patient receiving the transduced dendritic cells (e.g. PBMCs are collected from a patient, cultured such that immature dendritic cells are produced and transduced with the viral vectors of the present invention). Such transduced dendritic cells may be lypholized or otherwise stored until the patient comes in for treatment. In particular embodiments, the transduced cells are stored in a vial marked with the patient's identification information.

In some embodiments, the present invention provides methods comprising; a) providing; i) dendritic cells, and ii) a composition comprising an adenoviral vector, wherein the adenoviral vector comprises: A) an adenoviral capsid, wherein the adenoviral capsid comprises subgroup B adenoviral capsid fibers selected from the group consisting of Ad11, Ad14, Ad16, Ad21, Ad34, Ad35, and Ad50; and B) a nucleic acid molecule, wherein the nucleic acid molecule comprises a retrogen cassette sequence encoding a retrogen protein, wherein the retrogen protein comprises; I) an antigen protein, 11) a leader sequence linked to the N-terminal of the antigen protein, and III) a cell-binding domain linked to the C-terminal of the antigen protein; and b) contacting the dendritic cells with the composition at a MOI of at least 5 (i.e. 5 plaque forming units (pfu) per cell) under conditions such that the retrogen protein is expressed by at least 30% of the dendritic cells thereby generating retrogen-expressing dendritic cells wherein the antigen protein is presented by the retrogen-expressing dendritic cells as a MHC class-I antigen and a MHC class-II antigen.

In certain embodiments, the retrogen protein is expressed by at least 35% or at least 55% of the dendritic cells when the contacting is conducted at a MOI of 5-10. In particular, embodiments, the retrogen protein is expressed by at least 70% of the dendritic cells when the contacting is conducted at a MOI of 10-100 (See, FIG. 2). In other embodiments, the retrogen protein is expressed by at least 90% of the dendritic cells when the contacting is conducted at a MOI of 100-500 (See Ad11 in FIG. 2).

In some embodiments, the contacting occurs ex vivo. In particular embodiments, the method further comprises, before step b) collecting cells from a subject (e.g. human) such as monocytes, and culturing the cells (e.g. with GM-CSF or IL-4) to produce immature dendritic cells. In some embodiments, dendritic cells are collected directly from the subject. In some embodiments, PBMCs are collected from the subject via leukophoresis or other suitable method. The collected cells may then be subject to PBMC elutriation and the pure monocyte fraction collected. These collected cells may then be cultured such that immature DCs result.

In other embodiments, the contacting occurs in vivo. In particular embodiments, the compositions comprising the viral vectors of the present invention are administered to a subject such that at least a portion of the subject's dendritic cells are transduced. In preferred embodiments, the viral vectors of the present invention are administered to a patient (or dendritic cells comprising the viral vectors of the present invention are administered to a patient) under conditions such that the symptoms of the disease of the patient are reduced or eliminated. Preferably, the transduced dendritic cells in the subject results in stimulation of Th (e.g. CD4+ Th), and CTL (e.g. CD8+ CTL) response specific to the antigen expressed by the viral vectors of the present invention. In some embodiments, the contacting comprises administering the composition to a subject.

In some embodiments, the nucleic acid molecule further comprises left and right Ad ITR sequences. In other embodiments, the nucleic acid molecule further comprises an adenoviral capsid fiber encoding sequence. In certain embodiments, the nucleic acid molecule further comprises an Ad packaging sequence. In additional embodiments, the nucleic acid molecule lacks at least one adenoviral gene selected from the group consisting of L5, L2, TP, E1A, E2A, E4, E2A, Pol, IV, E1B, VA, LA, and E3 (e.g. E1 and/or E3). In other embodiments, the nucleic acid molecule lacks at least five adenoviral genes selected from the group consisting of L5, L2, TP, E1A, E2A, E4, E2A, Pol, IV, E1B, VA, L4, and E3 (e.g. E1, E3, E2A, E2B, and E4). In still other embodiments, the nucleic acid molecule lacks all or nearly all of the following adenoviral genes L5, L2, TP, E1A, E2A, E4, E2A, Pol, IV, E1B, VA, L4, and E3 (i.e. “gutless”).

In preferred embodiments, the adenoviral fibers are Ad11 fibers. In certain embodiments, the adenoviral capsid further comprises an Ad5 non-fiber capsid component. In some embodiments, the adenoviral capsid further comprises an Ad11 or Ad35 non-fiber capsid component. In still other embodiments, the nucleic acid molecule is inside the adenoviral capsid (See FIG. 1).

In certain embodiments, the antigen protein is a tumor associated antigen. In other embodiments, the tumor associated antigen is selected from MAGE, GAGE, DAGE, prostate-specific Ag, prostate-specific membrane Ag, tyrosinase, Gp100, α-fetoprotein, Ig idiotype, TCR, Bcr-abl fusion product, mutant p53, NY-ESO-1, Her-2/neu, and Muc-1. In preferred embodiments, the antigen protein is HBeAg or HBcAg.

In some embodiments, the cell binding domain comprises an Fc region (e.g. IgG1 Fc region). In other embodiments, the composition further comprises antigen presenting cells (APCs), such as dendritic cells (e.g. immature or mature dendritic cells). In certain embodiments, the composition further comprises an antigen presenting cells (e.g. dendritic cell) and the adenoviral vector is inside the APC (e.g. dendritic cell).

In some embodiments, the methods further comprise step c) administering the retrogen-expressing dendritic cells to a patient. In particular embodiments, the patient has a disease (e.g. infection, cancer, autoimmune disease, etc.). In certain embodiments, the patient has HBV-associated hepatocellular carcinoma or HBV infection. In particular embodiments, the contacting causes the dendritic cells to pass from an immature state to a mature state.

In some embodiments, the present invention provides methods comprising; a) providing; i) dendritic cells, and ii) a composition comprising an adenoviral vector, wherein the adenoviral vector comprises: A) an adenoviral capsid, wherein the adenoviral capsid comprises Ad11 capsid fibers; and B) a nucleic acid molecule, wherein the nucleic acid molecule comprises a transgene sequence encoding a protein of interest; and b) contacting the dendritic cells with the composition at a MOI of at least 5 under conditions such that the protein of interest is expressed by at least 55% of the dendritic cells thereby generating protein of interest-expressing dendritic cells.

In certain embodiments, the contacting causes the dendritic cells to pass from an immature state to a mature state. In other embodiments, the protein of interest is expressed by at least 70% of the dendritic cells when said contacting is conducted at a MOI of 10-100 (See FIG. 2). In some embodiments, the protein of interest is expressed by at least 90% of the dendritic cells when the contacting is conducted at a MOI of 100-500 (See FIG. 2).

In particular embodiments, the transgene sequence is a retrogen cassette sequence encoding a retrogen protein, wherein the retrogen protein comprises: i) an antigen protein, ii) a leader sequence linked to the N-terminal of the antigen protein, and iii) a cell-binding domain linked to the C-terminal of the antigen protein. In certain embodiments, the cell binding domain comprises an Fc region. In further embodiments, the antigen protein is presented by the protein of interest-expressing dendritic cells as a MHC class-I antigen and a MHC class-II antigen.

In some embodiments, the contacting occurs ex vivo. In other embodiments, the method further comprises, before step b) collecting cells from a subject (e.g. human) such as monocytes, and culturing the cells (e.g. with GM-CSF or IL-4) to produce immature dendritic cells. In some embodiments, dendritic cells are collected directly from the subject. In other embodiments, PBMCs are collected from the subject via leukophoresis or other suitable method. The collected cells may then be subject to PBMC elutriation and the pure monocyte fraction collected. These collected cells may then be cultured such that immature DCs result.

In other embodiments, the contacting occurs in vivo. In particular embodiments, the compositions comprising the viral vectors of the present invention are administered to a subject such that at least a portion of the subject's dendritic cells are transduced. Preferably, the transduced dendritic cells in the subject results in stimulation of Th (e.g. CD4+ Th), and CTL (e.g. CD8+ CTL) response specific to the antigen expressed the viral vectors of the present invention. In some embodiments, the contacting comprises administering the composition to a subject.

In some embodiments, the nucleic acid molecule further comprises left and right Ad ITR sequences. In other embodiments, the nucleic acid molecule further comprises an adenoviral capsid fiber encoding sequence. In certain embodiments, the nucleic acid molecule further comprises an Ad packaging sequence. In additional embodiments, the nucleic acid molecule lacks at least one adenoviral gene selected from the group consisting of L5, L2, TP, E1A, E2A, E4, E2A, Pol, IV, E1B, VA, L4, and E3 (e.g. E1 and/or E3). In other embodiments, the nucleic acid molecule lacks at least five adenoviral genes selected from the group consisting of L5, L2, TP, E1A, E2A, E4, E2A, Pol, IV, E1B, VA, L4, and E3 (e.g. E1, E3, E2A, E2B, and E4). In still other embodiments, the nucleic acid molecule lacks all or nearly all of the following adenoviral genes L5, L2, TP, E1A, E2A, E4, E2A, Pol, IV, E1B, VA, L4, and E3 (i.e. “gutless”).

In preferred embodiments, the adenoviral fibers are Ad11 fibers. In certain embodiments, the adenoviral capsid further comprises an Ad5 non-fiber capsid component. In some embodiments, the adenoviral capsid further comprises an Ad11 or Ad35 non-fiber capsid component. In still other embodiments, the nucleic acid molecule is inside the adenoviral capsid.

In certain embodiments, the antigen protein is a tumor associated antigen. In other embodiments, the tumor associated antigen is selected from MAGE, GAGE, DAGE, prostate-specific Ag, prostate-specific membrane Ag, tyrosinase, Gp100, α-fetoprotein, Ig idiotype, TCR, Bcr-abl fusion product, mutant p53, NY-ESO-1, Her-2/neu, and Muc-1. In preferred embodiments, the antigen protein is HBeAg or HBcAg.

In some embodiments, the method further comprises step c) administering the protein of interest-expressing dendritic cells to a subject. In particular embodiments, the subject has HBV-associated hepatocellular carcinoma or HBV infection.

In particular embodiments, the present invention provides compositions comprising nucleic acid sequences encoding, or encoding a part of, the viral vectors of the present invention. In some embodiments, the viral vectors are encoded by at least two nucleic acid sequence (e.g. a helper dependent adenoviral sequence and a helper adenoviral sequence or helper adenovirus that allows expression of the helper dependent adenoviral sequence that contains the transgene sequence, such a retrogen cassette).

In certain embodiments, the present invention provides kits comprising: a) a viral vector of the present invention or transduced dendritic cells comprising the viral vectors of the present invention; and b) instructions for using the viral vector or transduced dendritic cells to treat a disease in a subject or instructions for employing the viral vector or tranduced dendritic cells for scientific research. In some embodiments, the present invention provides cell lines stably or transiently transfected with nucleic acid sequences encoding the viral vectors of the present invention.

### DESCRIPTION OF THE FIGURES

FIG. 1 shows one embodiment of the present invention where the viral vector has Ad11 capsid fibers and is used to transduce an immature dendritic cell, resulting in a mature dendritic cell.

FIG. 2 shows the immature dendritic cell transduction efficicies of Ad5, Ad5/11 and Ad5/35 as described in Example 1.

FIG. 3 shows the effect of Ad5 and Ad5/11 infection on dendritic cell maturation as described in Example 1.

FIG. 4A shows the retrogen cassette sequence employed in Example 2. FIG. 4B shows the percent of GFP or retrogen expression in dendritic cells transduced with Ad5 and Ad5/11 as described in Example 2.

### Definitions

To facilitate an understanding of the invention, a number of terms are defined below.

As used herein, the terms “subject” and “patient” refer to any animal, such as a mammal like a dog, cat, bird, livestock, and preferably a human. In preferred embodiments, the subject has a disease amenable to treatment with dendritic cell based immunotherapy.

As used herein, the terms “transduction” or “infection” refer to a method of introducing viral DNA within a virus into a host cell (e.g. immature dendritic cell).

As used herein, the phrase “non-fiber capsid component” refers the capsid of a virus (e.g. adenovirus) minus the capsid fibers.

As used herein, the terms “an oligonucleotide having a nucleotide sequence encoding a polypeptide,” “polynucleotide having a nucleotide sequence encoding a polypeptide,” and “nucleic acid sequence encoding a peptide or protein” means a nucleic acid sequence comprising the coding region of a particular polypeptide. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc., or a combination of both endogenous and exogenous control elements.

Also, as used herein, there is no size limit or size distinction between the terms “oligonucleotide” and “polynucleotide.” Both terms simply refer to molecules composed of nucleotides. Likewise, there is no size distinction between the terms “peptide,” “protein,” and “polypeptide.” These terms simply refer to molecules composed of amino acid residues.

As used herein, the term “helper dependent viral DNA” or “gutted viral DNA” refers to viral DNA that codes for viral vectors that contain cis-acting DNA sequences necessary for viral replication and packaging, but generally no viral coding sequences (See U.S. Pat. No. 6,083,750, incorporated by reference). These vectors can accommodate up to about 36 kb of exogenous DNA and are unable to express viral proteins sufficient for replication. Helper-dependent viral vectors are produced by replication of the helper dependent viral DNA in the presence of a helper adenovirus, which alone or with a packaging cell line, supplies necessary viral proteins in trans such that the helper-dependent viral DNA is able to be replicated. Gutted vectors may be constructed as described in U.S. Pat. No. 6,083,750.

As used herein the term “helper viral DNA” refers to viral DNA encoding helper viral vectors, that are capable of providing, alone or with a packaging cell line, viral proteins in trans such that a gutted virus is able to replicate. For example, the helper virus may supply the capsid with the adenovirus subgroup B fibers. A “helper adenovirus” or “helper virus” refers to an adenovirus which is replication-competent in a particular host cell. The host may provide, for example, Ad gene products such as E1 proteins. The ‘helper virus’ is used to supply in trans functions (e.g., proteins) which are lacking in a second replication-incompetent virus (e.g. a gutted viral vector). Therefore, the first replication-competent virus is said to “help” the second replication-incompetent virus thereby permitting the propagation of the second viral genome in the cell containing the helper and second viruses. Helper virus may include a sequence capable of crippling helper virus replication in the presence of certain crippling agents. An example of a helper virus with a crippling sequence is the (+)lox(+)pol helper virus. The (+)lox(+)pol helper virus is an E1-, E3-deleted virus that can be negatively selected using Cre recombinase and carries an alkaline phosphatase reporter gene in its E3 region. The packaging signal, which consists of packaging elements I-V, is flanked by loxP sites in direct repeat orientation, allowing removal of the packaging signal in the presence of Cre (a crippling agent).

The term “virus” refers to obligate, ultramicroscopic, intracellular parasites incapable of autonomous replication (ie., replication requires the use of the host cell's machinery). Adenoviruses are double-stranded DNA viruses. The left and right inverted terminal repeats (ITRs) are short elements located at the 5′ and 3′ termini of the linear Ad genome, respectively, and are required for replication of the viral DNA. The left ITR is generally located between 1-130 bp in the Ad genome (also referred to as 0-0.5 mu). The right IR is located from ˜35,800 bp to the end of the genome (also referred to as 99.5-100 mu). The two ITRs are inverted repeats of each other.

As used herein, the term “gene of interest” or “transgene sequence” refers to a gene inserted into a vector or plasmid whose expression (expressing a protein of interest) is desired in a host cell. Transgene sequence include genes having therapeutic value as well as reporter genes. Preferably the trangene sequence encodes a protein of interest that is an antigen (e.g. tumor associated antigen).

As used herein, the term “treatment” refers to both therapeutic treatment and prophylactic or preventative measures. Those in need of treatment include those already with the disorder as well as those in which the disorder is to be prevented.

The phrase “under conditions such that the symptoms are reduced” refers to any degree of qualitative or quantitative reduction in detectable symptoms of any disease treatable by viral mediated dendritic cell based immunotherapy, including but not limited to, a detectable impact on the rate of recovery from disease (e.g., rate of weight gain), reduction in tumor size, or the reduction of at least one of the symptoms normally associated with the particular disease.

### DESCRIPTION OF THE INVENTION

The present invention provides compositions, methods and kits comprising viral vectors that may be used for performing immunotherapy. In particular, the present invention provides viral vectors having subgroup B adenoviral capsid fibers that are configured to express a transgene sequence in antigen presenting cells (e.g. dendritic cells) with a high transduction efficiency. Preferably, the transgene sequence is a retrogen cassette and the adenoviral capsid fibers are Ad11 fibers. The description of the invention is provided in the following sections below: I) Viral Vectors with subgroup B adenoviral fibers; II) Proteins of interest expressed by viral vectors; and III) Viral Vector Dendritic Cell Based Immunotherapy.

I. Viral Vectors with Subgroup B Adenoviral Fibers

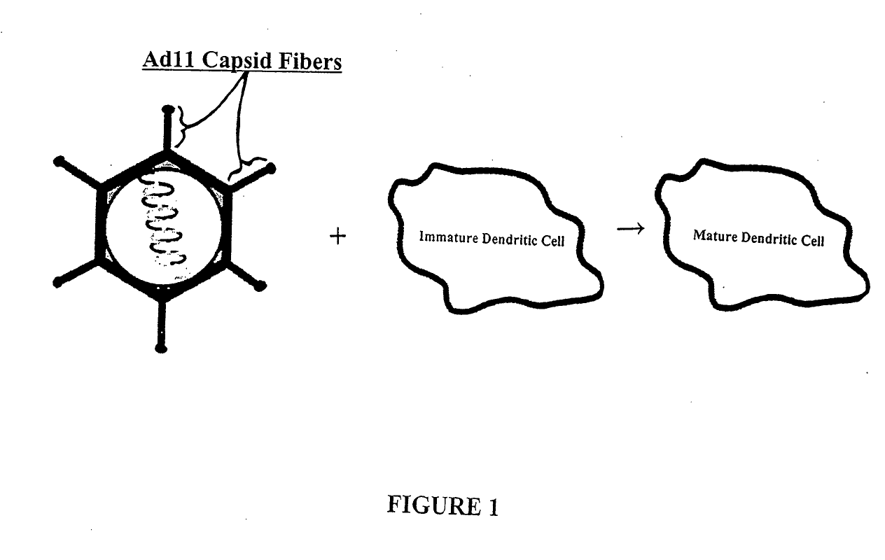

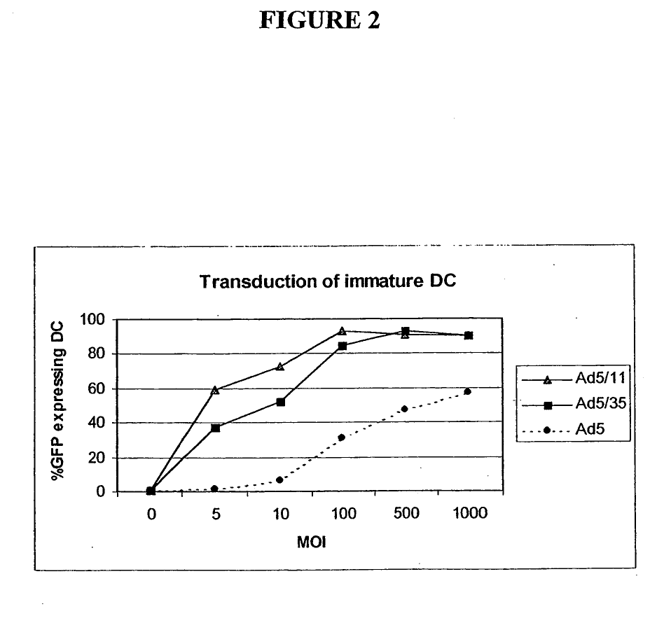

The viral vectors of the present invention possess subgroup B adenoviral fibers. For example, the subgroup B adenoviral fibers may be Ad11, Ad14, Ad16, Ad21, Ad34, Ad35, Ad50, or combinations thereof. FIG. 1 shows one embodiment of the present invention where the adenoviral capsid fibers are Ad11. The viral vectors of the present invention, with subgroup B adenoviral fibers (e.g. Ad11) have important advantages, such as increased tranduction efficiency for human dendritic cells, as well as causing maturation of immature human dendritic cells. Viral vectors with Ad11 fibers have shown to be particularly advantageous. For example, the transduction efficiency of Ad11 fiber based vectors showed dramatic improvements of Ad5 based vectors, as well as other viral vectors with subgroup B fibers, such as Ad35 (See, FIG. 2). The viral vectors of the present invention may be used to transduce dendritic cells at very low MOI as a result of the improved tropism. This allows the viral vectors to express a protein of interest (see below) in the dendritic cells at a low MOI. Such low MOIs allow for lower doses for transducing dendritic cells, and allows improvements to human immunotheapy protocols (see below).

Any type of viral vector may used with the present invention. For example, any type of non-enveloped DNA or RNA viruse already expressing subgroup B capsid fibers, or modified to express subgroup B capsid fibers (e.g. by deleting the normal fiber gene and replacing it with a group B capsid fiber) may be employed with the present invention. For example, in some embodiments, the viral vectors comprise adenoviral vectors with Ad35 or Ad11 fibers, while the rest of the adenoviral capsid is Ad5. These viral vectors are referred to as Ad5/11 or Ad5/35 adenoviral vectors. Techniques for generating such chimeric vectors have previously been described in the art (See, e.g., Shayakhmetov et al., J. of Virol., 74(6):2567-2583, 2000; Shayakhmetov and Lieber, J. of Virol. 74(22):10274-10286, 2000; and WO0073478, all of which are herein incorporated by reference in their entirities). Useful constructs are presented, for example, in FIG. 5A of Shayakhmetov et al. and FIGS. 16 and 18 of WO0073478. It is also noted that the sequence encoding the Ad11 fibers are found in the art (See, e.g. Genbank Accession No. L08231 and L08232; and Mei and Wadell, Virology, 194:453-462, 1993, herein incorporated by reference). Similar techniques may be used to produce other chimeric viral vectors.

As noted above, the present invention is not limited to any particular type of viral vector. A number of suitable vectors are known in the art including, but are not limited to, the following: adenoviral vectors; second generation adenoviral vectors; gutted adenoviral vectors; adeno-associated virus vectors; and lentiviral Vectors. All of these vectors may be modified (or already contain) subgroup B adenoviral fibers. Adenoviral vectors are preferred viral vectors of the present invention. Adenoviral vectors are described below in more detail.

1. Adenoviral Vectors

Adenoviral vectors are preferably used with the present invention. There are 51 Ad serotypes classified into subgroups A to F. Subgroup B adenoviral vectors already contain subgroup B adenoviral fibers, while groups A, and C-F can be modified to contain subgroup B adenoviral fibers. Self-propagating adenovirus (Ad) vectors have been extensively utilized to deliver foreign genes to a great variety of cell types in vitro and in vivo. “Self-propagating viruses” are those which can be produced by transfection of a single piece of DNA (the recombinant viral genome) into a single packaging cell line to produce infectious virus; self-propagating viruses do not require the use of helper virus for propagation. First generation adenoviral vectors are deleted for E1 and E3 genes.

2. Second Generation Adenoviral Vectors

In an effort to address the viral replication problems associated with first generation Ad vectors, so called “second generation” Ad vectors have been developed. Second generation may also be modified (or already contain) such that they have subgroup B adenoviral fibers. Second generation Ad vectors further delete the early regions of the Ad genome (E2A, E2B, and E4). Highly modified second generation Ad vectors are less likely to generate replication-competent virus during large-scale vector preparation, and complete inhibition of Ad genome replication should abolish late gene replication. Host immune response against late viral proteins is thus reduced [See Amalfitano et al., J. Virol. 72:926-933 (1998)]. The elimination of E2A, E2B, and E4 genes from the Ad genome also provide increased cloning capacity.

3. Gutted Adenoviral Vectors

“Gutted,” “gutless,” or helper dependent, Ad vectors contain cis-acting DNA sequences that direct adenoviral replication and packaging but do not contain viral coding sequences (See Fisher et al., Virology 217:11-22 (1996); Kochanek et al, Hum. Gen. Ther., 10:2451-9, 1999; and U.S. Pat. No. 5,994,132, all of which are herein incorporated by reference). Gutted vectors are defective viruses produced by replication in the presence of a helper virus, which provides all of the necessary viral proteins in trans. The helper virus may provide the sequences necessary produce the viral capsid. In the present invention, the helper virus can express capsids with subgroup B adenoviral fibers (e.g. Ad11 fibers). Since gutted vectors do not contain any viral genes, expression of viral proteins is not possible. Helper dependent viral production has been described in the art [See Hardy et al., J. Virol. 71:1842-1849 (1997) and Hartigan-O'Conner et al., J. Virol. 73:7835-7841 (1999)]. Gutted Ad vectors are able to maximally accommodate up to about 37 kb of exogenous DNA, however, 28-30 kb is more typical.

II. Proteins of Interest Expressed by Viral Vectors

As described above, the present invention is useful for the production of viral vectors with subgroup B adenoviral fibers (e.g. helper-dependent adenoviral vectors with subgroup B adenoviral fibers, such as Ad11 fibers). The viral vectors produced, in preferred embodiments, comprise a transgene sequence (heterologous nucleic acid sequence) encoding a protein of interest, such that the vectors may be useful for various applications (protein expression in vitro, therapeutic applications, immunotherapy with dendritic cells, etc). Suitable heterologous DNA sequences include, for example, nucleic acid sequences that encode a protein that is defective or missing in a recipient subject, or a heterologous gene that encodes a protein having a desired biological or therapeutic effect (e.g. an antibacterial, antiviral, or antitumor function). Other suitable heterologous nucleic acids include, but are not limited to, those encoding for proteins used for the treatment of endocrine, metaloic, hematologic, cardiovascular, neurologic, musculoskeletal, urologic, pulmonary, and immune disorders, including such disorders as inflammatory diseases, autoimmune disease, chronic and infectious diseases, such as AIDS, cancer, hypercholestemia, insulin disorders such as diabetes, growth disorders, various blood disorders including various enemias, thalassemias, and hemophilia; genetic defects such as cystic fibrosis, Gaucher's disease, Hurler's disease, adenosine deaminase (ADA) deficiency, and emphysema.

In preferred embodiments, the transgene sequence will code for an antigen protein. The antigen may include a native protein or protein fragment, or a synthetic protein or protein fragment or peptide. Examples of antigens include, but are not limited to, those that are capable of eliciting an immune response against viral or bacterial hepatitis, influenza, diphtheria, tetanus, pertussis, measles, mumps, rubella, polio, pneumococcus, herpes, respiratory syncytial virus, hemophilus influenza type b, chlamydia, varicella-zoster virus or rabies.

In other preferred embodiments, the antigen protein is a tumor or cancer associated antigen. For example, the protein antigen may include, but is not limited to, from MAGE-1 or MAGE-3 (for melanoma, lung, and colorectal cancer treatment), GAGE, DAGE, prostate-specific Ag (for treating prostate cancer), prostate-specific membrane Ag (for treating prostate cancer), tyrosinase (for treating melanoma), Gp100 (for treating melanoma), α-fetoprotein (for treating liver cancer), Ig idiotype (for treating B-cell NHL, myeloma), TCR (for treating T-cell NHL), Bcr-abl fusion product (for treating CAL), mutant p53 (for treating lung, colorectal, head and neck cancer), NY-ESO-1 (for treating melanoma and breast cancer), Her-2/neu (for treating breast, lung, and ovarian cancer), and Muc-1 (for treating pancreatic, lung, breast, and colorectal cancer).

In particularly preferred embodiments, the antigen is from the HBV virus. As noted above, HBV infection and HCC are major world wide health problems. The viral vectors of the present invention may comprise a transgene sequence encoding an HBV antigen, and be used to transduce dendritic cells (to treat patients with HBV infection or HCC). One example of an antigen from HBV is the hepatitis B core antigen (HBcAg). Another antigen from HBV is the HBeAg (sequences encoding these antigens are known in the art). For example, the coding sequence for HBcAg is found in genbank accession number M38594. The human hepatitis B virus is composed of an outer lipid envelope embedded with surface antigen proteins and an inner protein capsid that contains the viral genome. The icosahedral nucleocapsid is composed of multiple subunits of the Hepatitis B core antigen (HBcAg). A nonparticulated form of the HBcAg, the HBeAg, is secreted in the serum during HBV infection. HBeAg shares amino acids 1-149 of HBcAg and contains an N-terminal extension of 10 amino acids encoded by the precore sequence of the precore-core (HBeAg) gene. Both HBcAg and HBeAg are highly immunogenic in most hosts and prime humoral and T-cell responses in mice and macaques. In certain preferred embodiments, within the HBeAg gene, the arginine-rich amino acid residues (aa 150-180) at the HBeAg C-terminus are deleted.

In other preferred embodiments, the transgene sequence in the viral vectors of the present invention are retrogen cassette sequences. The Retrogen technology uses a Retrogen cassette as the transgene in a vector. The Retrogen cassette is composed of a sequence encoding an antigen (e.g. a tumor associated antigen) with a cell-binding domain sequence (for receptor mediated internalization) at the C-terminus of the antigen, and a leader sequence/secretory sequence at the N-terminal (allowing secretion) of the antigen. This technology has been previously described (See, e.g, You et al., Cancer Research, 61:197-205, 2001; and WO03025126 to Chen et al., both of which are herein incorporated by reference in their entirities).

Because the antigen-presenting pathway to MHC-class I is distinctively different from that to MHC-II, it is difficult for an antigen to be presented to both MHC-I and II by DCs. For example an intracellular antigen expressed by transduced DCs can be efficiently processed and presented by MHC-I but not by MHC-II. On the other hand, secretory protein expressed from transduced DCs cannot be presented by MHC-I. The retrogen cassette technology allows presentation of antigens to both MHC-I and MHC-II and potently activates Th, CTL and B-cells (See, You et al., above). The coding sequence of an antigen is modified with an N-terminal leader sequence allowing for secretion and with, for example, an Fc fragment of IgG allowing for antigen-reuptake and endocytosis into DCs via the Fc-γ-receptor. The modified gene is then transduced into DCs to produce and secrete the fusion proteins (termed “retrogen” for its retrograde transport/internalization), which can be taken up by DCs via receptor-mediated endocytosis, processed in the endosomal pathway and presented by DCs as exogenous for MHC-II presentation to induce CD4+ Th cells. The internalized antigens can also be presented to MHC-I to directly activate CTLs (cross-priming). The interaction of Fc with its FcγR activates DCs by upregulating surface molecules and cytokines involved in antigen presentation. The combination of the adenovirus subgroup B fiber expressing viral vectors of the present invention and retrogen cassettes marks a powerful combination for effecting DC transduction and human immunotherapy.

III. Viral Vector Dendritic Cell Transduction Based Immunotherapy

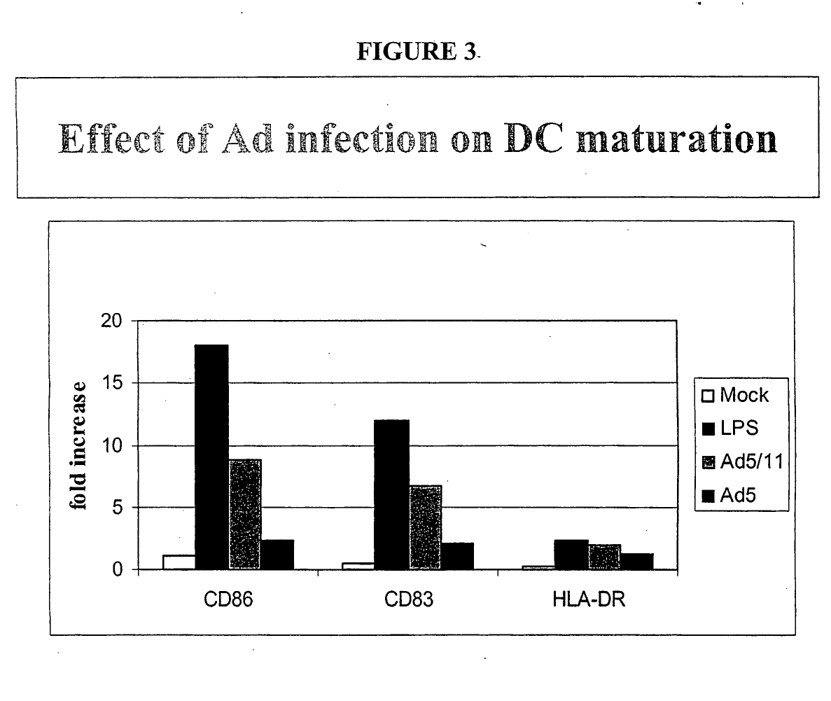

The viral vectors of the present invention are preferably used to transduce dendritic cells (e.g. human dendritic cells) such that the dendritic cells activate a Th, CTL, and/or B cells response in a patient in need of treatment. The viral vectors of the present invention with adenoviral subgroup B fibers have been found to transduce dendritic cells at low MOIs (see FIG. 2) and to activate maturation (see, FIG. 3). In preferred embodiments, the viral vectors of the present invention comprise Ad11 capsid fibers, and are used to transduce immature dendritic cells (see, FIG. 1). Below are additional details on dendritic cells and immunotherapy.

Dendritic cells (DCs) are specialized antigen-presenting cells that play a pivotal role as a bridge for the innate, cellular and humoral immune responses. In the body, DCs are located strategically at the interface of potential pathogen entry sites. They capture antigens and migrate into secondary lymphoid tissues, where they can activate both helper T-cells (Th) and cytotoxic T-lymphocytes (CTLs). They also interact with B-cells and probably NK cells. DCs develop from bone marrow (myeloid) progenitors (CD34+) into peripheral blood progenitors (CD11c+), which migrate to various peripheral tissues through interaction with their selectins (e.g. CD62L), adhesion molecules (e.g. LFA-1, VLA-4, CD44, CLA), and chemokine receptors (e.g. CCR1, -5, -6). In their immature state, DCs efficiently take up antigens. Upon antigen uptake, and exposure to natural stimuli, including certain infectious agents, inflammatory cytokines, or triggering of their CD40 receptor, DCs are mobilized and migrate via the draining lymphatics to the peripheral lymphoid organs, where they encounter T-cells. During this migratory process DCs mature into cells that are specialized in presenting high numbers of peptide/MHC class I and II complexes in a rich co-stimulatory context. With DC activation and migration, antigen uptake activity and the associated antigen receptors are down regulated, resulting in a switch from antigen uptake to antigen presentation. Antigens are processed through both the class I and II MHC pathways, which are required for the activation of CD8+ CTLs and CD4+ Th cells, respectively. CD4+ cells produce cytokines/co-stimulatory molecules, which in turn, stimulate CTL and B-cells. CTLs are capable of directly killing tumor cells. The mature stage of DCs ends by apoptotic death in the lymph nodes.

The present invention provides methods for DC-mediated immunotherapy, and in particular, immunotherapy of cancer using the viral vectors described herein. Anti-tumor vaccination strategies utilizing plain antigen proteins/peptides often lead to poor vaccination or even tolerization of T-cells. By harnessing the capacity of DCs to present tumor antigens to T-cells, DCs can serve as the centerpiece of an immunotheraputic approach to cancer. Two main strategies have been used for DC-mediated immunotherapy of tumors; coculturing (pulsing) DCs with antigen protein and genetic modification of DCs. DCs have been pulsed with class I-restricted synthetic peptides or proteins, or with natural peptides eluted from autologous tumors. This category of approaches also includes exposure to whole tumor lysates or apoptotic bodies as well as the fusion of tumor cells with DCs. Strategies for genetic modification of DCs are based on antigen DNA or RNA delivery into DCs using viral or non-viral vectors (See, Nouri-Shirazi et al., Immunol. Lett. 74:5-10, 2000, herein incorporated by reference). DCs engineered for expression of a given antigen provide important advantages over antigen-pulsed DCs in terms of i) continuous production of large amounts of therapeutic protein for a relatively long time period, which is important for presentation in complex with MHC-class I, that has a relatively short turnover rate; ii) endogenous production and processing of antigens which potentially allows for better access to the MHC class I pathway and presentation of multiple and unidentified epitopes (in contrast, the use of peptides is dependent on the knowledge of the HLA haplotype of the patient, which restricts the usage of peptide epitopes of any given antigen); and iii) the potential of stimulation of Th1 and Th2 as well as T-and B-cell responses. The feasibility of DC-mediated immunotherapy of cancer has been demonstrated in a series of pilot studies (see Nestle, Oncogene, 19:6673-9, herein incorporated by reference). For example, in antigen/peptide-loaded DC-based clinical trials, complete and partial responses in patients with malignant melanoma, lymphoma, renal cell carcinoma, and prostate cancer have been obtained at frequencies, which have not been observed previously with established modes of cancer vaccination (Dannull et al., Onkologie, 23:544-551, 200, herein incorporated by reference).

The viral vectors of the present invention (with adenoviral subgroup B fibers, such as Ad11) may be used to deliver transgenes (e.g. retorgen cassettes) to dendritic cells. Generally, non-viral, ex vivo gene delivery into DCs is inefficient. Among the viral vectors, recombinant retroviruses, Herpes simplex virus, vaccinia virus, and adenoviruses have been used for DC transduction (all of these viruses may be modified to contain adenoviral subgroup B fibers in accordance with the present invention). Transduction rates of cultured DCs with onco-retroviruses are very low (Westermann et al., Gene Ther., 5:264-71, 1998, herein incorporated by reference). The viral vectors of the present invention, however, have high transduction efficiencies and therefore are optimally suited for dendritic cell transduction.

For immunotherapy application, the DCs of the present invention may be transduced ex vivo or in vivo. For example, PBMCs may be collected from a patient with cancer via leukophoresis or other suitable method. The monocytes may then be purified by elutriation or other suitable method. The pure monocyte fraction may then be induced to differentiate into dendritic cells by culturing with GM-CFS, IL-4 or other suitable agent. The immature dendritic cells may then be transduced with the viral vectors of the present invention that may comprise, for example, a tumor associated antigen, to yield mature dendritic cells (See FIG. 1). The dendritic cells may then be administered to the patient to treat the cancer or other disease in the patient. The dendritic cells optimally activate both a Th, and CTL response (and B cell response) in the patient. The dendritic cell transduction may also be carried out by directly injecting the viral vectors of the present invention into a patient such that the dendritic cells in the patient are transduced.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: M (molar); mM (millimolar); nM (nanomolar); pM (picomolar); mg (milligrams); μg (micrograms); pg (picograms); ml (milliliters); μl (microliters); ° C. (degrees Celsius); OD (optical density); nm (nanometer); BSA (bovine serum albumin); and PBS (phosphate-buffered saline solution).

### Example 1

### Viral Vectors with Ad11 and Ad35 Fibers Transduce Dendritic Cellss with High Efficiency and Active Dendritic Cell Maturation

This example describes assays used to test the ability of adenoviral vectors with either Ad11 or Ad35 caspid fibers to transduce human dendritic cells. This example also tests the ability of these same viral vectors to activate maturation in human dendritic cells.

In this example, human dendritic cells (DCs) were generated by ex vivo differentiation. In particular, human DCs were generated by in vitro differentiation from myeloid CD34+ progenitors (from cadaveric bone marrow) as well as from CD34+ progenitors or CD14+ monocytes isolated from peripheral blood after mobilization with G-CSF. Standard protocols for ex vivo differentiation of CD34+ progenitors with GM-CSF, IL-4, f13-ligand, and TNF were employed (See, Ferlazzo et al., J. Immunol. 163:3597-604, 1999, herein incorporated by reference).

The adenoviral vectors used in this example were first generation adenovirus Ad5 vectors configured for expressing GFP with either Ad11 or Ad35 fibers (instead of Ad5 fibers). These viral vectors are referred to as Ad5/11 or Ad5/35 adenoviral vectors. Techniques for generating such chimeric vectors have previously been described in the art (See, e.g., Shayakhmetov et al., J. of Virol., 74(6):2567-2583, 2000; Shayakhmetov and Lieber, J. of Virol. 74(22):10274-10286, 2000; and WO0073478, all of which are herein incorporated by reference in their entirities). Useful constructs are presented, for example, in FIG. 5A of Shayakhmetov et al. and FIGS. 16 and 18 of WO0073478. It is also noted that the sequence encoding the Ad11 fibers are found in the art (See, e.g. Genbank Accession No. L08231 and L08232; and Mei and Wadell, Virology, 194:453-462, 1993, herein incorporated by reference).

Ad5/35 and Ad5/11 vectors, as well as Ad5 vectors, were used for gene transfer into the immature human DCs. Transduction was carried out at various MOIs for 3 hours at 37 degrees Celsius and GFP expression was analyzed by flow cytometry 24 hours post infection. The results of this transduction show that 37% and 59% of human CD11c+DCs were transduced at an MOI of 5 (pfu/cell) of Ad5/35 and Ad5/11, respectively. At the same MOI, less than 1% of myeloid DCs were transduced with the standard Ad5 vector. These results are shown in FIG. 2. Importantly, maximal transduction efficiency with Ad5/35 and Ad5/11 vectors (85-95%) was achieved at an MOI of 100, while only 58% transduction was achieved with the highest Ad5 dose test (MOI 1000). Among a number of promoters to drive transgene expression from Ad5/35 and Ad5/11 vectors only the CMV promoter, but not the MSCV or RSV promoters, conferred GFP expression in DCs, indicating that these promoters are not active in human DCs. Ad5/11 and Ad5/35 vectors cannot transduce mouse DCs. Compared to Ad5 vectors, the transduction kinetics with Ad5/35 and Ad5/11 vectors were greatly enhanced, resulting in highly efficient gene expression within hours after exposure to virus. Gene expression remained high during LPS-induced ex vivo maturation of DCs.

The effect of Adenovirus infection on expression of DC maturation markers was also examined. Immature DCs were infected with Ad5-, Ad5/11-, and Ad5/35-CMV-GFP (MOI 10) or treated with LPS. DC surface markers were analyzed by flow cytometry 24 hours later. FIG. 3 shows these results. FIG. 3 reports these results as the increase in surface markers compared to immature DCs. The mock was incubation with only the virus dilution buffer. As these results in FIG. 3 show, Ad5/35 and Ad5/11 infection also triggered DC maturation as assessed based on the increase in CD86, CD83, and HLA-DR expression (FIG. 3). In contrast, infection with Ad5 vectors at an MOI of 10 did not induce a significant elevation in maturation markers. Furthermore, DC maturation (at a level similar to that shown in FIG. 3) was also induced by incubation of immature DCs with empty Ad5/11 capsids indicating that the interaction with the putative Ad11 receptor and/or particle uptake is sufficient to stimulate signaling pathways that lead to DC maturation. For comparison, upregulation of surface markers was assessed after addition of LPS, which is routinely used to induce DC maturation. The ability of Ad vectors with Ad11 or Ad35 fibers to efficiently tansduce DCs at very low MOIs together with induction of maturation, indicates that these vectors should be very useful tools in human immunotherapy (e.g. DCs transduced in vivo, or DCs transduced ex vivo and then injected into a patient in need of treatment).

### Example 2

### Expression of HBeAg-Retrogen in HeLa Cells Transduced by Ad5 and Ad5/11 First-Generation Vectors

This example describes the expression of HBeAg-Retrogen in HeLa cells transduced by Ad5 and Ad5/11 first-generation vectors. This example combines the use of vectors with Ad11 fibers with the “Retrogen” technology. The Retrogen technology uses a Retrogen cassette as the transgene in a vector. The Retrogen cassette is composed of a sequence encoding an antigen (e.g. a tumor associated antigen) with a cell-binding domain sequence (for receptor mediated internalization) at the C-terminus of the antigen, and a leader sequence/secretory sequence at the N-terminal (allowing secretion) of the antigen. This technology has been previously described (See, e.g, You et al., Cancer Research, 61:197-205, 2001; and WO03025126 to Chen et al., both of which are herein incorporated by reference in their entirities).

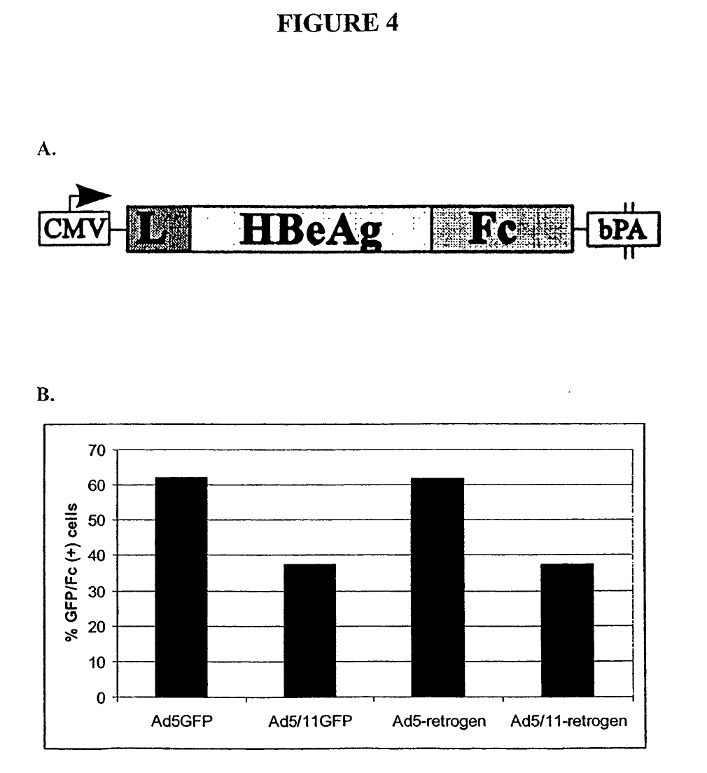

In this example, the antigen encoding sequence of the Retrogen cassette was the HBeAg antigen from the human hepatitis B virus (HBV). The human hepatitis B virus is composed of an outer lipid envelope embedded with surface antigen proteins and an inner protein capsid that contains the viral genome. The icosahedral nucleocapsid is composed of multiple subunits of the Hepatitis B core antigen (HBcAg). A nonparticulated from of the HBcAg, the HBeAg, is secreted in the serum during HBV infection. HBeAg shares amino acids 1-149 of HBcAg and contains an N-terminal extension of 10 amino acids encoded by the precore sequence of the precore-core (HBeAg) gene. Both HBcAg and HBeAg are highly immunogenic in most hosts and prime humoral and T-cell responses in mice and macaques. In this example, within the HBeAg gene, the arginine-rich amino acid residues (aa 150-180) at the HBeAg C-terminus that are cleaved during HBV infection were deleted. The HBeAg used is derived from the “ayw” HBV subtype. The truncated (secreted) HBeAg (with its own leader sequence, L) was linked to a cell-binding domain (human IgG Fc cDNA) at its C-terminus for receptor-mediated internalization. The HBeAg gene was placed under the control of the CMV promoter, which is known to be active in DCs. Transcription is terminated by the bovine growth hormone polyadenylation signal (bPA). This Retrogen cassette is shown schematically in FIG. 4A.

In this example, HeLa cells were infected with first-generation Ad5 and Ad5/11-based vectors (see Example 1) containing a GFP or the HBeAg Retrogen expression cassette at a MOI of 10. The HeLa cell were analyzed by flow cytometry for GFP expression or with FITC-labelled anti-Fc 48 hours post-infection for HBeAg expression. Cells were not permealized. The results are shown in FIG. 4B. Similar levels of (intracellular) GFP expression and retrogen expression indicate that the retrogen can be readily detected in the membrane of the live cells. It is noted that HeLa cells express both Ad5 and Ad11 receptors.

### Example 3

### Expression of HBeAg-Retrogen in Dendritic Cells Transduced by Ad5 and Ad5/35 First Generation Vectors

This example describes the expression of the HBeAg Retrogen transduced by Ad5 and Ad5/35 first generation vectors. The Retrogen cassette employed is described in Example 2, and the Adenoviral vectors are described in Example 1. In this example, immature CD11c positive DCs were infected with Ad5 and Ad5/35. Ad5/35 infected DCs showed enhanced expression of HBeAg compared to the Ad5 infected cells. Infection of immature DCs was performed as described in Example 1. Ad5 and Ad5/35 HBeAg infected DCs were then incubated with antigen loaded autologous peripheral T cells. Enhanced T cell proliferation and secretion of the Th1 cytokine IFN-γ was seen in T cells incubated with Ad5/35 HBeAg infected DCs compared to Ad5 HBeAg infected cells. We also found that HBeAg-specific CTL activity was stronger in settings with DCs transduced with the retrogen expressing Ad.5/35 vector compared to peptide-loaded DCs.

Taken together, these results indicate that group B pseudotyped adenoviruses enable ore efficient DC transduction, maturation and stimulation of antigen specific immune responses at lower, not toxic MOI's.

### Example 4

### Human Immunotherapy Employing Viral Vectors with Subgroup B Capsid Fibers

This example describes performing human immunotherapy with viral vectors with subgroup B capsid fibers. In particular, this example describes treating a human with immunotherapy by transducing immature dendritic cells in, or from, a human patient with hepatocellular carcinoma (HCC) due to HBV infection.

A human patient with HCC may be treated with an adenoviral vector with subgroup B capsid fibers configured to express a Retrogen cassette. For example, an adenoviral vector with Ad11 fibers that contains a Retrogen cassette for expressing HBeAg may be used to transduce a human patient's immature DCs. DC transduction may occur ex vivo. DCs from the patient may be isolated from the patient and then transduced by the adenoviral vector. Alternatively, the adenoviral vector may be injected into the patient near a concentration of DCs (e.g. lymph nodes) or near a tumor. The transduction (in vivo or ex vivo) can occur at a low MOI, such as MOI of 100 or a MOI of 10. The transduced immature DCs will mature and present both MHC type I and type II antigens to the T cells and B cells in the patient. It is expected that Th, CTL and B cells responses will be mounted against the HCC tumors in the patient as well as HBV infection in the patient. This immunotherapy should reduce or eliminate the symptoms of HCC and HBV infection.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, medicine, and molecular biology or related fields are intended to be within the scope of the following claims.

We claim:

1. A composition comprising an adenoviral vector, wherein said adenoviral vector comprises:
a) an adenoviral capsid, wherein said adenoviral capsid comprises subgroup B adenoviral capsid fibers selected from the group consisting of Ad11, Ad14, Ad16, Ad21, Ad34, Ad35, and Ad50; and
b) a nucleic acid molecule, wherein said nucleic acid molecule comprises a retrogen cassette sequence encoding a retrogen protein, wherein said retrogen protein comprises;
i) an antigen protein,
ii) a leader sequence linked to the N-terminal of said antigen protein, and
iii) a cell-binding domain linked to the C-terminal of said antigen protein.

2. The composition of claim 1, wherein said adenoviral capsid fibers are Ad11 fibers.

3. The composition of claim 1, wherein said antigen protein is a tumor associated antigen.

4. The composition of claim 1, wherein said antigen protein is HBeAg.

5. The composition of claim 1, further comprising dendritic cells.

6. The composition of claim 1, wherein said composition further comprises a dendritic cell, and wherein said adenoviral vector is inside said dendritic cell.

7. A method comprising;
a) providing;
i) dendritic cells, and
ii) a composition comprising an adenoviral vector, wherein said adenoviral vector comprises:
A) an adenoviral capsid, wherein said adenoviral capsid comprises subgroup B adenoviral capsid fibers selected from the group consisting of Ad11, Ad14, Ad16, Ad21, Ad34, Ad35, and Ad50; and
B) a nucleic acid molecule, wherein said nucleic acid molecule comprises a retrogen cassette sequence encoding a retrogen protein, wherein said retrogen protein comprises;
I) an antigen protein,
II) a leader sequence linked to the N-terminal of said antigen protein, and
III) a cell-binding domain linked to the C-terminal of said antigen protein; and
b) contacting said dendritic cells with said composition at a MOI of at least 5 under conditions such that said retrogen protein is expressed by at least 30% of said dendritic cells thereby generating retrogen-expressing dendritic cells, wherein said antigen protein is presented by said retrogen-expressing dendritic cells as a MHC class-I antigen and a MHC class-II antigen.

8. The method of claim 7, wherein said retrogen protein is expressed by at least 35% of said dendritic cells when said contacting is conducted at a MOI of 5-10.

9. The method of claim 7, wherein said retrogen protein is expressed by at least 70% of said dendritic cells when said contacting is conducted at a MOI of 10-100.

10. The composition of claim 7, wherein said contacting occurs ex vivo.

11. The method of claim 7, wherein said adenoviral capsid fibers are Ad11 fibers.

12. The method of claim 7, wherein said antigen protein is a tumor associated antigen.

13. The method of claim 7, wherein said antigen protein is HBeAg.

14. The method of claim 7, further comprising step c) administering said retrogen-expressing dendritic cells to a patient.

15. The method of claim 14, wherein said pateint has HBV-associated hepatocellular carcinoma or HBV infection.

16. A method comprising;
a) providing;
i) dendritic cells, and
ii) a composition comprising an adenoviral vector, wherein said adenoviral vector comprises:
A) an adenoviral capsid, wherein said adenoviral capsid comprises Ad11 capsid fibers; and
B) a nucleic acid molecule, wherein said nucleic acid molecule comprises a transgene sequence encoding a protein of interest; and
b) contacting said dendritic cells with said composition at a MOI of at least 5 under conditions such that said protein of interest is expressed by at least 55% of said dendritic cells thereby generating protein of interest-expressing dendritic cells.

17. The method of claim 16, wherein the contacting causes said dendritic cells to pass from an immature state to a mature state.

18. The method of claim 16, wherein said protein of interest is expressed by at least 70% of said dendritic cells when said contacting is conducted at a MOI of 10-100.

19. The method of claim 16, wherein said protein of interest is expressed by at least 90% of said dendritic cells when said contacting is conducted at a MOI of 100-500.

20. The method of claim 16, wherein said contacting occurs ex vivo.

21. The method of claim 16, wherein said protein of interest is HBeAg.

22. The method of claim 16, further comprising step c) administering said protein of interest-expressing dendritic cells to a subject.

23. The method of claim 22, wherein said subject has HBV-associated hepatocellular carcinoma or HBV infection.
